(19) United States
(12) Patent Application Publication
(10) Pub. No.: US 2019/0290525 A1
(43) Pub. Date: Sep. 26, 2019

(54) Upper Extremity Radial Artery Procedure Support Device And Associated Warming Sleeve

(71) Applicant: Robo-Rad By Dr. K L.L.C., Las Vegas, NV (US)

(72) Inventor: Collins Kwarteng, Las Vegas, NV (US)

(73) Assignee: Robo-Rad By Dr. K L.L.C., Las Vegas, NV (US)

(21) Appl. No.: 16/304,483

(22) PCT Filed: May 26, 2017
(86) PCT No.: PCT/US2017/034668

§ 371 Date: Nov. 26, 2018

Related U.S. Application Data
(60) Provisional application No. 62/341,890, filed on May 26, 2016.

(51) Int. Cl. A61G 13/12; A61B 17/00; A61F 7/02; F16B 47/00

(52) U.S. Cl. CPC A61G 13/1235; A61B 17/00234; A61G 13/129; A61F 2007/0029; F16B 47/00; A61B 2017/00292; A61F 7/02

(57) ABSTRACT

A multi-jointed, upper extremity radial artery procedure support device and associated warming sleeve is disclosed. The support device may function like a human arm and is designed to support both right-sided and left sided interventions. The multi-jointed radial arm support device may allow flexion and extension of a patient's arm at the patient's associated elbow and wrist. The arm-warming sleeve may apply gentle warmth to the patient's arm during a procedure to induce vasodilation of the radial artery, and eliminate a need for vasoactive drugs during radial artery procedures.

### FIELD OF THE INVENTION

The present invention relates to an upper extremity radial artery procedure support device, and associated warming sleeve, such as for use when performing a trans-radial cardiac catheterization process on a patient

Background of the Invention

The history of invasive cardiology dates back to 1711 when Stephen Hales placed catheters into the right and left ventricles of a living horse. Cardiac catheterization in a human however was first performed in 1929, when Werner Forssman catheterized himself by inserting a catheter through his left antecubital vein, advancing it all the way to his right atrium and confirming it under a chest X-ray. Over the following decades this procedure underwent a series of metamorphoses. In 1958, an interventional radiologist by the name of Dr. Charles Dotter devised methods to visualize the coronary anatomy.

Until the 1950's, access to an arterial or venous system for catheterization typically required a physical cut down. The percutaneous approach widely used today, was developed in 1953 by a radiologist Sven-Ivar Seldinger. Building on work previously done by Drs. Melvin Judkins and Charles Dotter, Dr. Andreas Gruentzig performed the first successful percutaneous trans-luminal coronary angioplasty (PTCA) on a human in Zurich on Sep. 16, 1977, by using a balloon-tipped catheter to dilate a severely narrowed coronary artery. His pioneering work ushered into the mainstream, the field of percutaneous coronary intervention or PCI.

Historically coronary angiography and intervention have been done via a percutaneous access of the femoral artery using the modified Seldinger technique (where the anterior wall of the artery is punctured by a hollow needle). Through this femoral access, a port with a one-way-valve, called a sheath, is placed, and through the sheath, a diagnostic or interventional catheter is advanced to cannulate the coronary arteries over a guide wire, with the patient laying very still in a supine position (lying flat on his/her back, horizontally with face and torso facing generally upwards). Although this route of accessing the femoral artery typically guarantees an ability to access a large caliber vessel, the femoral artery, it comes with potentially significant drawbacks, which includes, but are not limited to; a risk of significant bleeding, infection due to the close proximity to the groin/perineum, as well as significant discomfort to the patient since the patient may have to lay down for over four to six hours after the procedure, to prevent catastrophic bleeding. The time spent in a hospital bed also means that costs attributed to the procedure may also be much higher, to the patient and/or payor, who gets billed for the time the patient spends in the hospital. Hence different means of accessing arteries in order to reach and cannulate coronary arteries were investigated.

The trans-radial access to perform diagnostic cardiac catheterization was first introduced by Campeau in 1989, and later adapted by Kiemeneij and Laaman in 1997 for therapeutic procedures of coronary angioplasty. At the dawn of the 21^{st} century, the trans-radial route gained in popularity, and now rivals the trans-femoral access especially in Europe and Asia. In the United States however, the shift to trans-radial artery access, has been relatively slow.

Advantages for performing a coronary artery angiography or intervention via the trans-radial route, include, but are not limited to, relatively short procedural time and reduced risk of vascular complication including bleeding, to mention a few. But perhaps the most important reason the trans-radial method has become so popular, is the comfort it provides the patient.

After a trans-radial procedure, the patient is usually able to sit up in bed and ambulate or walk around in approximately one hour, compared to the four to six hours in a supine position after a trans-femoral method. It is time-saving, and less costly to the patient and the insurance companies. Hospitals are able to free up a hospital bed. These advantages have led to the acceptance of the use of the trans-radial access as a method of coronary interventions, including during acute ST-Elevation myocardial infarction interventions.

To perform a trans-radial procedure, an interventional cardiologist uses the radial artery in the wrist (left or right) of a patient as the entry point for the catheter. The cardiologist (typically always standing on the right side of the patient on the operating table), threads a thin hollow catheter through the patient's network of arteries in the arm and into the chest in a retrograde fashion, eventually reaching the patient's aortic root. Accordingly the patient needs to be prepared and positioned in a supine position. The patient's arm needs to be immobilized in the anatomical position (arms to the sides of the body with the palms facing forward), and with the hand fully extended at the wrist, to expose the flat part of the forearm where the radial artery can be easily palpated and accessed. To achieve this, devices such as radial arm-boards have been proposed, and designed, to offer support for the patient's arm during the procedure. Most of these arm-supports are designed to only support the patients right arm. In certain cases however, the procedure needs to be done from the left arm, or through the left radial artery. Because the operator typically always stands on the right side of the patient who is laying supine on the operating table, if the procedure needs to be done via the left arm (the side opposite the operator), it requires the left arm to be presented in such a way that the arm comes across the abdomen, to place the left wrist at the patient's lower abdomen. That way, the operator doesn't have to strain him or herself to reach across the left wrist. For patient and physician comfort, as well as to provide the most optimal left arm support, there is a need for a paradigm shift in how a patient's left arm is supported.

Canulating the radial artery may also lead to vasoconstriction. As a result there is typically a need to infuse a systemic vasoactive drug through the sheath in the radial artery, to reduce such vasoconstriction. These vasoactive drugs may also cause systemic reduction in blood pressure, and would be contraindicated in a radial artery procedure of a critical patient with low blood pressure to begin with. The application or gentle warmth may be applied to the patient's arm to prevent such vasoconstriction.

A comprehensive look at the plethora of arm boards currently on the market, shows that most of them just create a flat surface for the arm to rest on, with a few accessories such as soft cushions, or even rolled cloths under the wrist. These flat boards or surfaces are typically hard, and hence are relatively uncomfortable, defeating the purpose of a trans-radial procedure, and most of them do not even offer a support mechanism for a left sided procedure. There is no device on the market known to the inventor which uses the application of gentle heat to the patient's arm to induce vasodilation of the radial artery during radial artery interventions.

Other devices such as a “radial access table” have tried to overcome the drawback of the flat surfaces by creating a separate table that can be set up to attach to the main procedure table. This radial table and the likes of it, touts its ability to swivel into many positions as its advantage. See for example Schaeffer, U.S. Pat. No. 6,821,288, and Neri, Published US Patent Application No. 2016/0008199. However this may not be particularly practical, since this table may be heavy and cumbersome to setup and disassemble, especially during acute coronary interventions, where an ability to quickly setup and get the patient re-vascularized is extremely vital. The radial table, and the likes of it, may also not overcome the flat surface disadvantage of the flat arm boards. As a result, Dr. Kwarteng conceived of, and invented the present radial arm support device, as well as an arm-warming sleeve to support both right-sided and left sided radial artery mediated coronary angiograms/interventions, as described below.

### SUMMARY

In accordance with one aspect of the invention, a multi-jointed radial arm support device is provided which functions like a human arm and is designed to support both right-sided and left sided interventions.

The multi-jointed radial arm support device may allow flexion and extension of a patient's arm at the patient's elbow.

The multi-jointed radial arm support device may allow flexion and extension of the wrist.

In accordance with another aspect of the invention, an arm-warming sleeve may be provided, which may apply gentle warmth to the patient's arm during a procedure to induce vasodilation of the radial artery, and eliminate a need for vasoactive drugs during radial artery procedures.

The arm-warming sleeve may be used together with the multi-jointed radial arm support device, or used independently.

Other features and advantages of the present invention should become apparent from the following description of the preferred embodiment, taken in conjunction with the accompanying drawings, which illustrate, by way of example, principles of the invention.

### DESCRIPTION OF THE FIGURES

For a more complete understanding of the disclosure, reference should be made to the following detailed description and accompanying drawings, wherein:

FIG. 1 illustrates a right-sided view of one embodiment of an arm support device in accordance with the present invention, shown in an optimal functional position—forearm flexed at the elbow and the hand extended at the wrist;

FIG. 2 illustrates an inside view of the right-sided device from an opposite side of the bed, shown in an optimal position.

FIG. 3 illustrates a top-down view of the device with the observer standing at the right shoulder;

FIG. 4 illustrates a view from the underside of the table showing how the vice attaches to the procedure table with a cork-screw mechanism and the suction cup;

FIG. 5 illustrates a view of the vice attachment to the table an observer looking from the head;

FIG. 6 illustrates a left-sided view of the left arm support device;

FIG. 7 illustrates a view of the left arm support from the foot of the procedure table, and also showing the same vice attachment from the left side of the table;

FIG. 8 illustrates a view of the left arm support device from the left shoulder of the patient;

FIG. 9 illustrates an inside view of the left arm support device from the right side of the table;

FIGS. 10a-10c illustrate an open, unwrapped and unapplied views of the one embodiment of the arm-warming sleeve;

FIGS. 11a-11b illustrate a fully wrapped and applied view of the arm-warming sleeve;

FIGS. 12a-12d illustrate different views of the left arm support as applied to the patient on the procedure table; and

FIGS. 13a-13f illustrate different views and orientations of the right arm support device as applied to a patient on the operating table.

### DESCRIPTION OF THE PREFERRED EMBODIMENTS

While this invention is susceptible of embodiment in many different forms, there will be described herein in detail, specific embodiments thereof with the understanding that the present disclosure is to be considered exemplifications of the principles of the invention and is not intended to limit the invention to the specific embodiments illustrated.

Due to deficiencies and imperfections of previously known radial arm-boards and tables, and the likes of it, currently on the market, Dr. Collins Kwarteng, a practicing interventional cardiologist, conceived of the present invention, to provide a radial arm-board support system that overcomes the deficiencies of the previously known arm-boards. The radial arm-board support system, was born out of Dr. Kwarteng's dissatisfaction he experienced when he personally used some of the previously known arm-boards on the market. Performing 80% of his diagnostic and interventional cases via the trans-radial access, Dr. Kwarteng had become very familiar with many of the devices already on the market, and he had not liked any of them. Dr. Kwarteng had come to realize that not only were these flat-board devices uncomfortable for the patients, but they were also not optimal for the operator (the cardiologist or radiologist) performing the procedure. Therefore, with all the things he disliked about the other devices he had been using, Dr. Kwarteng decided to invent a unique, robotic radial arm-board support system that functions like the patient's own arm.

Dr. Kwarteng's radial arm-board support system is provided with six main objectives in mind: 1) to create a device that mimics the patient's own upper extremity anatomy in a natural position; 2) to provide relative comfort to the patient during the procedure; 3). To offer the operator a unique work environment in which to work; 4) to provide relatively easy setup and disassembly; 5) to offer durability so as to be able to withstand the rough handling of a typical Cath-lab environment; and 6) to use the application of gentle heat to the patient's arm through an arm-warming sleeve, to induce thermal-mediated vasodilation, and thereby reduce or eliminate the need for vasoactive drugs during radial artery interventions. Dr. Kwarteng's radial arm-board support system and arm-warming sleeve concept, simulates the patient's own anatomy, and uses joints similar to a robotic arm, and designed by a practicing interventionalist, to optimize arm support for both right as well as left radial procedures. The arm-warming sleeve provides thermal-mediated vasodilation.

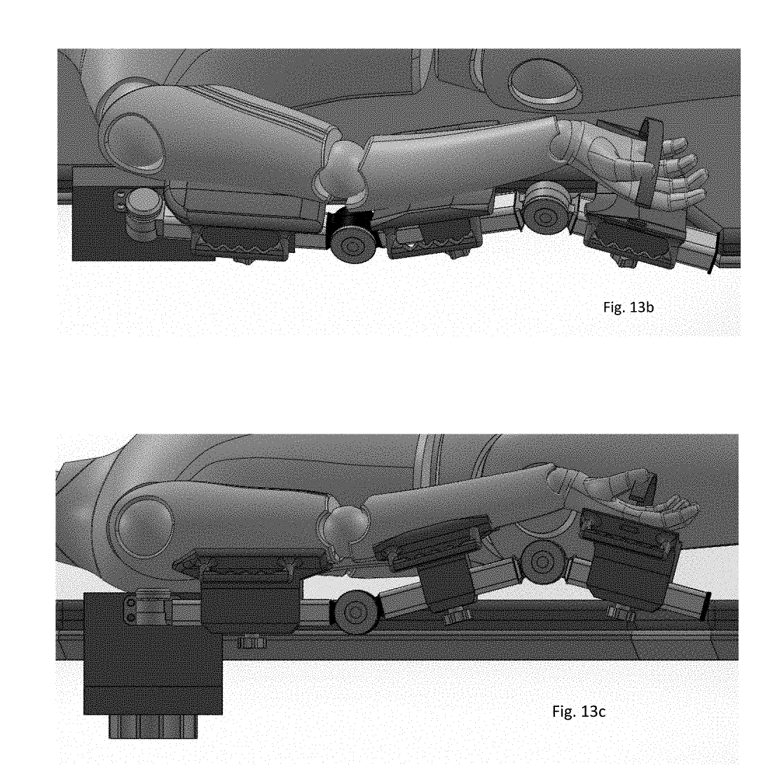

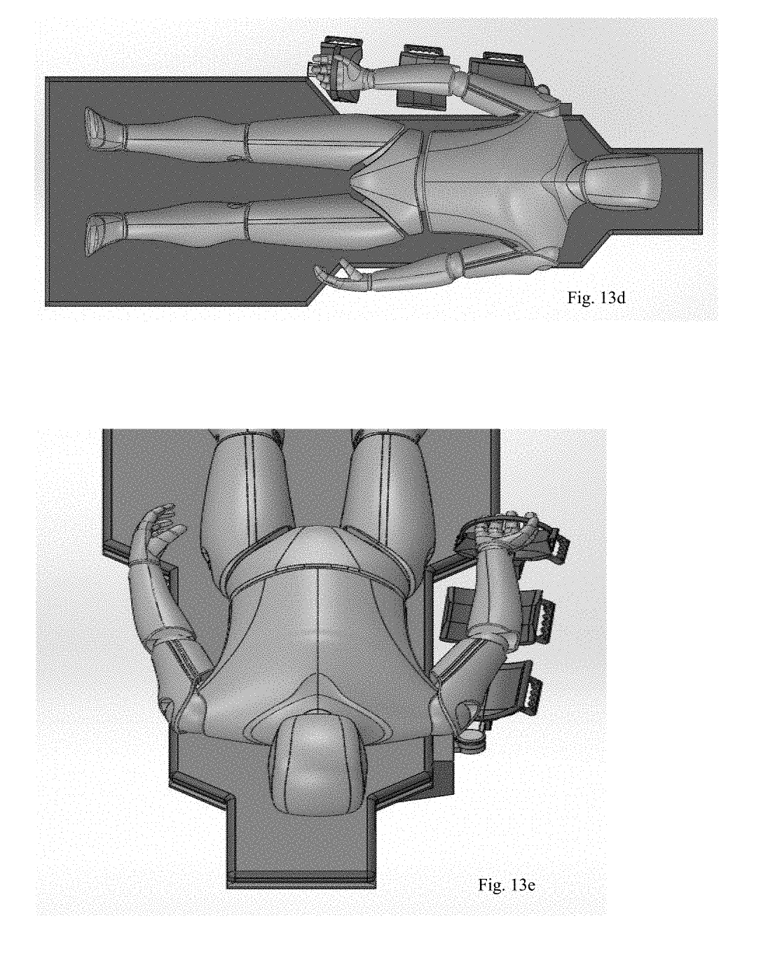

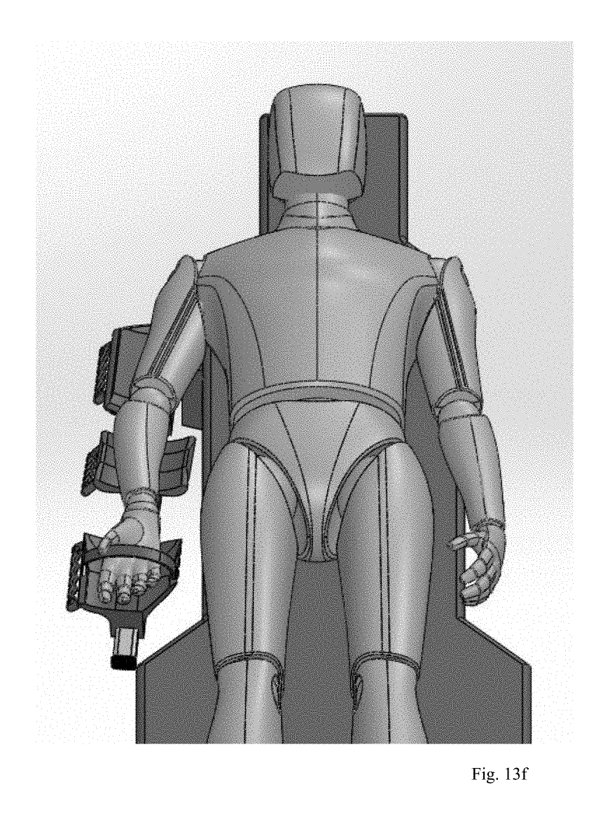

In accordance with the present invention, a radial arm-board support system, generally designated 50, provides the look of a robotic arm, with a firm attachment to the procedure table, via a heavy-duty suction cup or a “vice grip” system (FIG. 1, 100), and with articular joints at the shoulder (FIG. 1, 103), elbow (105) and wrist (105). The shoulder joint is firmly attached to the underside of a conventional procedure table proximate the patient's right or left shoulder. See FIGS. 13a-13e to see how the right arm support is supposed to look like in a real world. It is designed to mimic or look like the patient's arm, and thus be able to offer optimum support for the patient's arm in its natural anatomical position. The portion of the device that is in physical contact with the patient's arm may be made of a durable, medical grade, crack-resistant plastic, in a shallow “u”-shaped armrests (107), lined with a soft gel-foam padding to offer a very comfortable resting place for the arm. There may be three of these armrests, one for each of the patient's upper arm, forearm and hand. The shoulder joint (103) may move in the horizontal plane (through an angle of up to 180+ degrees), to help abduct or adduct the patient's arm, and to help place the patient's arm as close as possible to the side of the body, in a position optimal for both the patient and doctor. The elbow and wrist joints (105) place the patient's arm in an optimal position for both the patient and the operator, through flexion at the elbow and extension at the wrist.

The three joints are connected to each other through three durable rectangular carbon-fiber tubes (104), which may run down the backs of the “u”-shaped arm rests, providing it with unmatched support.

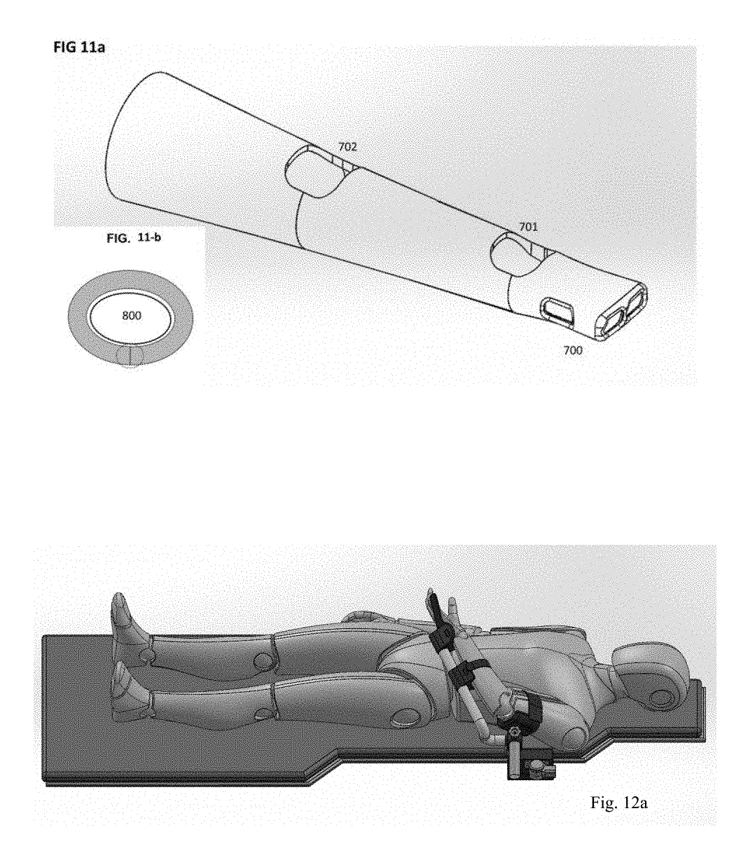

Because the conventional flat-board devices currently on the market are placed under the patients mat, and are not adjustable, such devices only support the patient's arm at a very low level relative to the hip/groin area where femoral access is obtained, which is the most ideal level where the hand is supposed to be. The articulated joints in the elbow of the present invention especially overcomes this disadvantage seen in the flat board supports, by allowing for flexion at the elbow to present the patient's arm to the hip area.
An additional feature of the present invention includes a heated arm sleeve. (See FIGS. 10a-10c and FIGS. 11a-11b) The heated arm sleeve may wrap around the patient's arm from the top of the patient's biceps and triceps to the wrist leaving an open space at the flat part of the forearm, and also in the antecubital fossa where the radial artery can be accessed and intravenous lines can be placed. The heated arm sleeve may be fastened into place via conventional, cooperating male and female Velcro strips sewn along long edges of sleeve. The heated arm sleeve may be made from three layers of cloth or other such material. The outer and inner layers may sandwich the middle layer where the heat is applied. The heating mechanism in the middle layer may be from heated electric coils, from flushing warm water through the middle layer, or by blowing warm air through the middle layer. The heat applied may be regulated to be safe so as to avoid burns, or other discomfort, to the patient.

The heated sleeve helps vasodilate the arteries, and minimize the rate of arterial puncture-induced vasoconstriction. Ordinarily as part of the routine trans-radial procedure, an intra-arterial cocktail of vasoactive drugs (typically verapamil and nitroglycerine) are administered via a sheath in the artery, to keep the radial and brachial arteries vasodilated. By slowly and carefully warming up the arm through the heated sleeve, it is the expectation that, the arteries will remain relatively relaxed, and will be less likely to shrink-down in size during arterial puncture. This should reduce medication administration during trans-radial procedures as well as the number of aborted trans-radial procedures due to severe radial artery vasoconstriction.

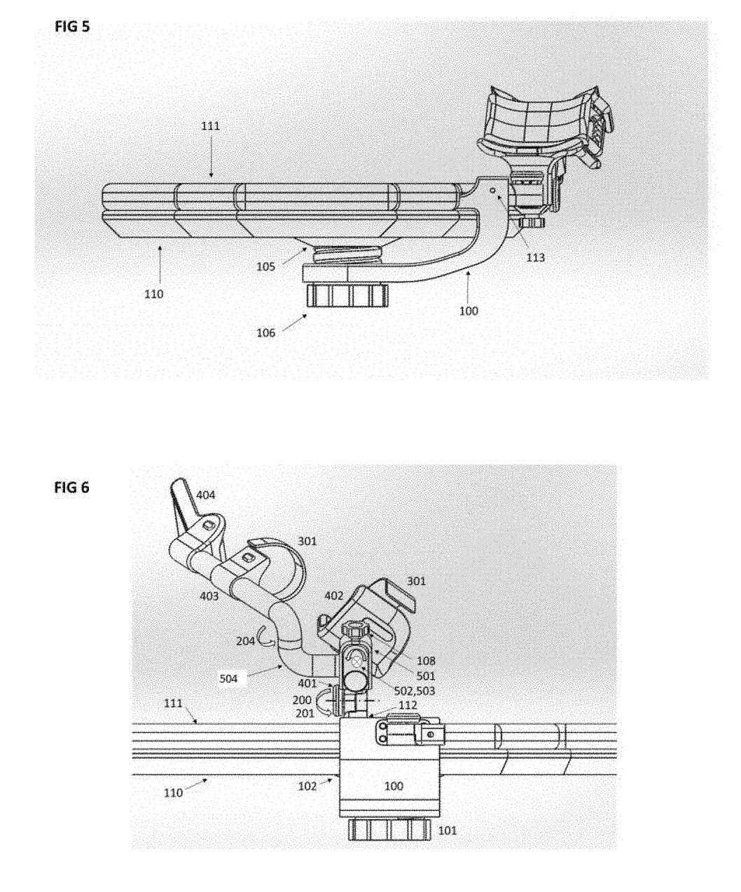

The present radial arm-board support system is provided to be relatively light in weight, and relatively easy to setup and take down. It can be relatively effortlessly mounted and easily taken down by even the tiniest of personnel. The device may also be used from either the right (FIGS. 1-5 and 13a-13f) or left (FIGS. 6-9 and 13a-d) side of the patient.

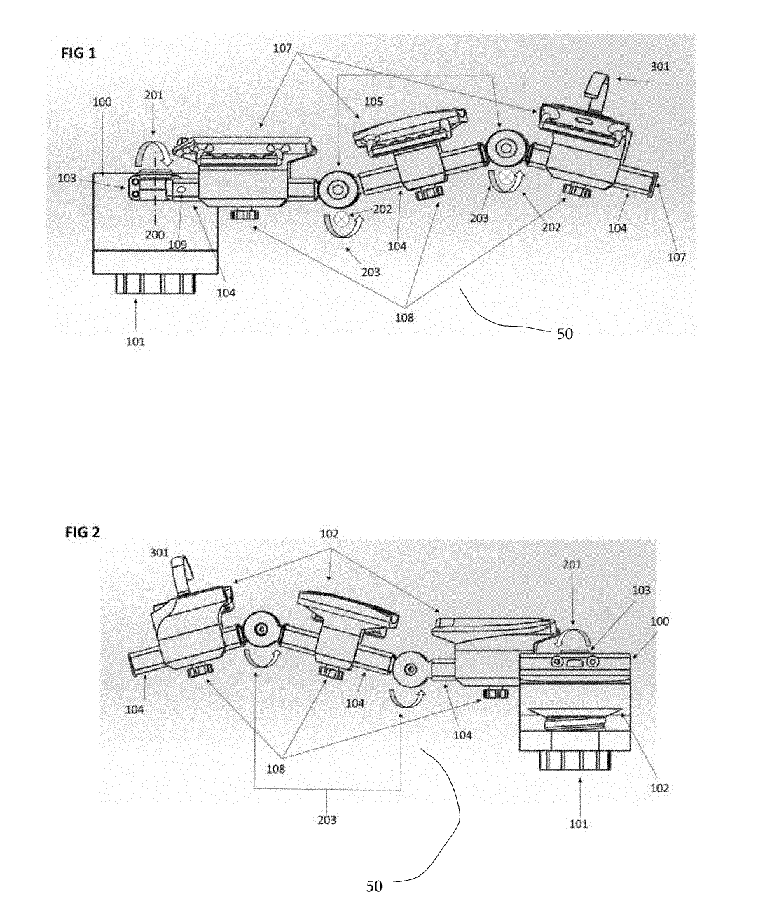

Referring to FIGS. 1 and 2, a side view (from the physician's perspective) of the robotic radial arm support device 50, in accordance with the present invention, is illustrated. As shown in FIG. 1, the robotic radial arm support device 50 is illustrated in the right-side arrangement. The robotic radial arm support device 50, in the right-side arrangement, may connect to a conventional surgical table with the use of a vise 100, which may consist of an adjustable, corkscrew mechanism 101 attached to a vacuum cup 102 with a conventional suction pump apparatus (not shown). Attached to the vise 100 along its outer side is an adjustable locking flange and armature joint 103 attached to the vise 100 via its flange, and attached to a square carbon fiber tube 104 via its armature. An axis 200 labeled as a hashed line describes rotational motion 201, labeled as a curved arrow, of the armature portion of the joint 103 along a horizontal plane, which is perpendicular to the page. In a safe locked mode, the horizontal rotational motion along this plane is only permitted when a button atop the joint 103 is pressed downwards so that the locking buttons are flush with the armature portion of the joint 103. This allows the operator to abduct or adduct the rest of the arm support to a desired location around the shoulder joint. Releasing the button on top of the joint locks it in place.

Attached to the right of the leftmost carbon fiber tube 104, is an adjustable, locking double armature joint 105, which moves along the axis 202, labeled as a circle inscribed with an “X”, that moves in the vertical plane only. The curved arrow describes the rotational movement 203 that the joint 105 permits which is along the plane of the page. Again, the rotational movement 203 is allowed when the button atop the joint 105 is pressed downwards so that the button is flush with the armature of the joint. Attached to the armature portion of the leftmost double armature joint 105 is another carbon fiber tube 104. At the end of this tube 104 exists another adjustable locking double armature joint 105, which may permit movement also in a vertical plane as the previous joint 105. The rightmost carbon fiber tube 104 connects to the rightmost double armature locking joint 105 on its left. A plug 106 may be located at the end of this carbon fiber tube 104 to prevent materials from entering the open end of the tube 104. Support structures 107 may rest around each carbon fiber tube 104. Each support structure 107 may be specially designed to support a patient's upper arm, lower arm and hand (left to right on figure). Each support structure 107 may move along the length of its respective carbon fiber tube 104 as desired until locked in place by screwing in each support structure's stopping mechanism 108. A spring-loaded button 109 may be provided so that all objects to the right of that release point can be removed from the leftmost carbon fiber tube 104 when pressed, while the device is pulled.

FIG. 2 is a side view of the right-side arrangement opposite of the physician. This view shows the inner portion of the vise 100, its corkscrew mechanism 101 which adjusts for surgical table thickness, and its vacuum 102 atop said corkscrew mechanism.

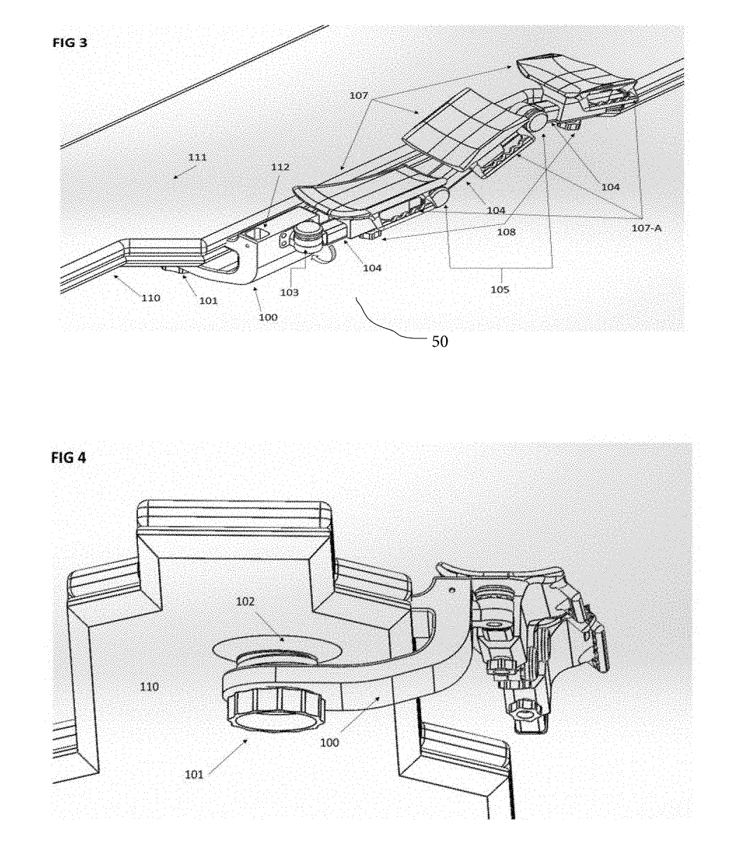

FIG. 3 is an angled view of the right-side arrangement. The vise may connect to the table between the surgical bed 110 and the surgical bed cushion 111 via the vise's upper lip. The corkscrew mechanism 101 on the vise is turned until the vacuum 102 contacts the underside of the surgical bed 110. The support structures 107 can be seen in a more detailed way customized so that each can fit the contour of the human arm. Handles 107-A, on each support structure 107 may allow for easy adjustment of the device while a joint button 103/104 is being pressed. The opening 112 where the left-side arrangement connects can also be seen.

FIG. 4 is a bottom-up view of the right-side arrangement connected to the surgical bed 110. The corkscrew mechanism may be rotated until its vacuum cup 102 meets the surgical bed 110. Once the vacuum comes in contact, the vacuum pump (not shown) may be pumped multiple times to apply sufficient suction at the vacuum cup 102.

FIG. 5 is a side-view looking from the head of the surgical table. The hole on the vise 112 (FIG. 3) shows where the left-side arrangement snaps in. A spring-loaded button locks the left-side arrangement in this location.

FIG. 6 shows the left side assembly of the robotic arm support device, seen from the left side of the table, opposite the physician. The vise 100 may be secured to the surgical table 110 via the corkscrew mechanism 101 and vacuum 102. The lip of the vise 100 is located between the surgical table 110 and the surgical table cushion 111. A double armature joint 401 may be snapped into the vise at location 112 via one of its armatures. A better image of 112 while not in use, is illustrated in FIG. 3. This adjustable locking, double armature joint 401 may rotate about a generally horizontal axis 200. A button atop the joint 401 allows movement. The second end of the armature of the joint 401 may be connected to a double-female adapter 502. A generally round tube 501, such as of a carbon fiber, may be attached to this adapter 502. The tube 501 may slide freely through the hole of the double-female adapter 502, as indicated by the “X”, and may rotate within the hole as indicated by the curved arrow 503, while the stopper 108 connected to the double-female adapter 502, is in the unlocked position. At the end of the tube 501, closest to the physician, is the upper-arm support 402, with an attached Velcro arm strap 301. Attached to the double-female adapter 502 is a flexible, adjustable, snake mechanism 504 with a flexible protective cover. This snake mechanism extends behind the arm of the patient allowing the forearm support 403 with its Velcro arm strap 301 and the hand support 404 to move along it. The hand support 404 has a bar at its end where the hand grasps onto. These supports 403/404 can both move along, and rotate about the adjustable snake mechanism 502.

The left-sided support device is different from the right-sided arm support (FIGS. 1-5) for a significant reason. In radial artery-mediated coronary angiograms or interventions, the operator always stands on right side of the table (the right side of the patient when the patient is laying on his or her back on the operating table and facing the ceiling). From that position, a procedure through the patient's right radial artery/right arm is simple, as the patient's right arm is directly in front of the operator. However, if the procedure is done through the left arm, the left arm needs to be presented across the torso such that the access site (the left radial artery), is in the patient's groin area. This way, the operator doesn't have to reach all the way across the patient to get to the patient's left arm. Without an arm support purposely built for the left arm, the operator will need to depend on the patient to continue to hold his or her arm in position throughout the procedure. Since the procedure is typically performed with the patient under moderate sedation, expecting a patient to hold their arm in place is not practical.

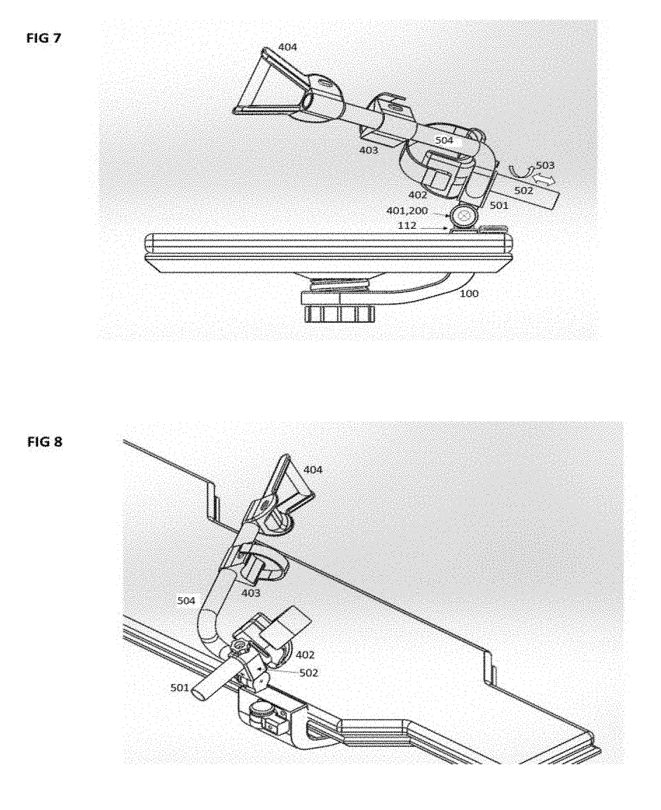

FIG. 7 is a side view from the perspective of the foot of the surgical bed. This view displays a more detailed view of the movements of the left-side arrangement. The double armature joint 401, which may rotate along the plane of the page, along its axis 200, denoted with an “X”, going into the page, is connected to the vise 100 at the vise's opening at location 112. As the double armature joint 401 rotates, the double-female adapter 501 does so as well. The carbon fiber tube 501 moves through a hole in the double-female adapter. This hole allows the tube to move along the hole, and rotationally within the hole as indicated by the curved and double-headed arrows 503. The adjustable, snake mechanism is also attached to the side of the double-female adapter. Additional views are provided of the support structures 402-404.

FIG. 8 is a top-down view of the left-side arrangement. This view shows a more detailed look into the hand support 404. The wrist sits on the arched section of the hand support 404, and the hand sits inside the hole, with the fingers wrapped around the bar.

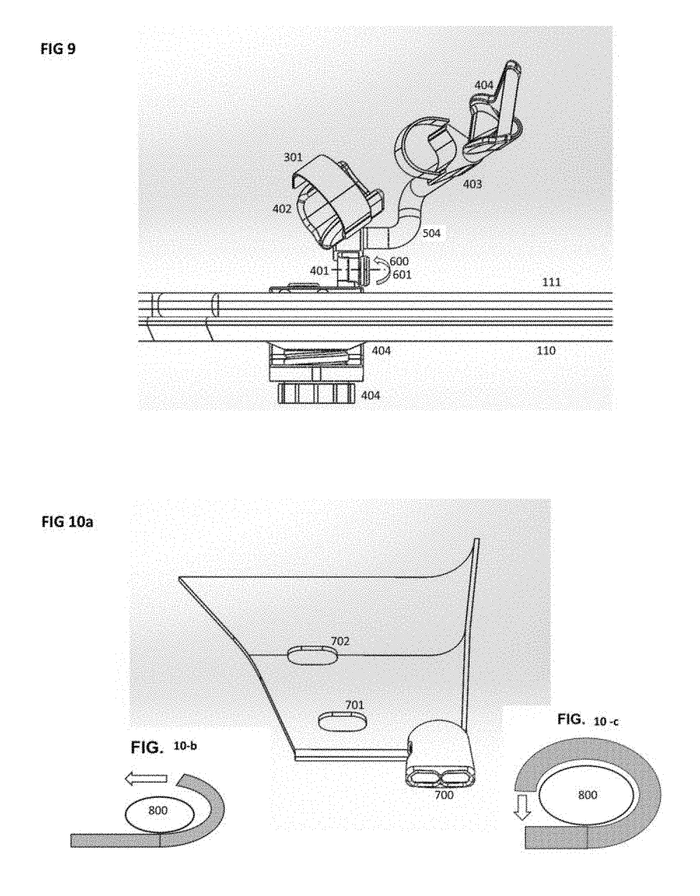

FIG. 9 is the left-side arrangement from the physician's perspective. More detail of the upper arm support 402 is seen here. The upper arm sits in the lip of the support structure 402, then set in place using a Velcro strap 301.

FIG. 10a illustrates an open and unwrapped view of one embodiment of an arm-warming sleeve in accordance with the invention. Openings at 701 and 702 allows access to the radial arteries and veins in the antecubital fossa when fully applied (FIG. 11a). The opening at 700 may allow the fingers of the hand and the thumb to secure the sleeve, when applied, and prevent the sleeve from pulling up. The arm-warming sleeve is made from three layers of a fabric, such as cloth or other appropriate material purposely designed with a heating mechanism sandwiched in the middle. The heating mechanism may be electrical, warmed water, an activated chemical reaction or via the circulation of heated air blown through an apparatus in the middle layer.

FIGS. 10b and 10c illustrate how the sleeve may be applied by wrapping the sleeve around the patient's arm, 800. The sleeve may be fastened into place via conventional male and female Velcro strips sewn along the long edges of the sleeve.

FIG. 11a illustrates a top-down view of the arm-warming sleeve in its closed arrangement. The fingers and thumbs of the hand of the patient fits through the holes 700 at the distal end of the sleeve. The openings 701 and 702 may allow for access to the patient's radial artery and antecubital veins respectively.

FIG. 11b illustrates a fully wrapped or deployed sleeve around the patient's arm 800.

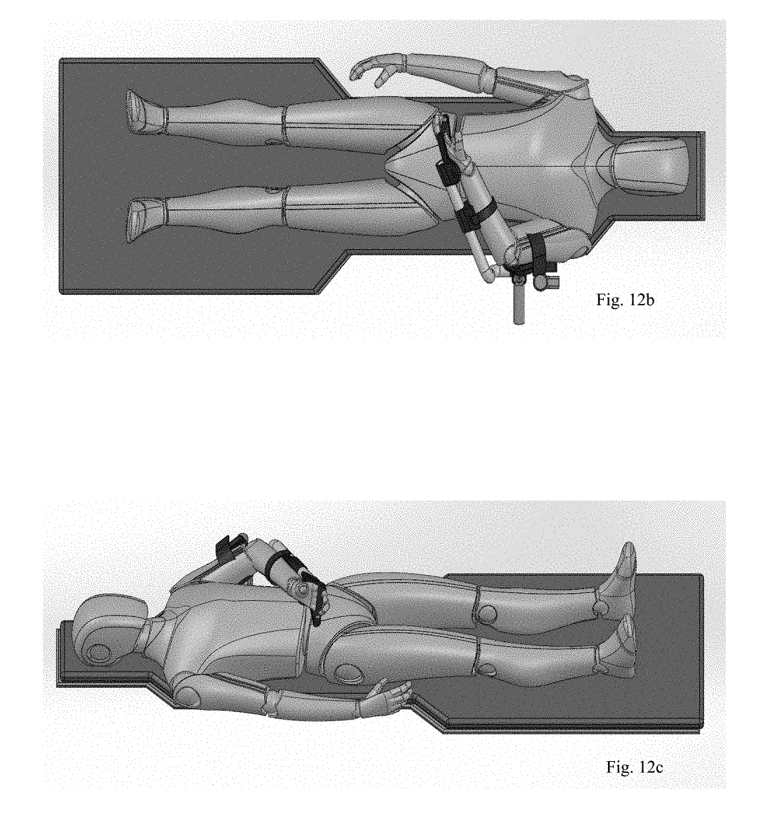

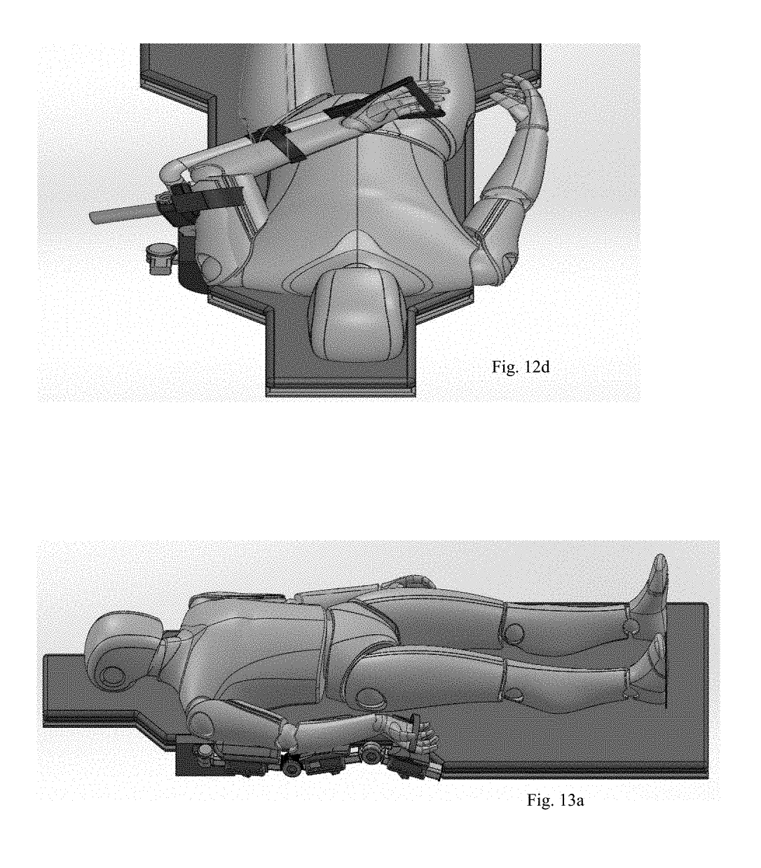

FIGS. 12a-12d illustrate how the left arm support looks like in the real world when fully applied. Different views are shown to better help conceptualize the idea of the left sided support.

FIGS. 13a-13f illustrate a real-world application of the right arm support. The different views of FIGS. 13a-13f are provided to better conceptualize the right-sided support system.

It is to be understood that this disclosure is not intended to limit the invention to any particular form described, but to the contrary, the invention is intended to include all modifications, alternatives and equivalents falling within the spirit and scope of the invention.

I claim:

1. For a patient positioned on a table in a supine position, a radial artery procedure support device for supporting the patient's arm during a radial artery procedure, the arm comprising an upper arm portion, a lower arm portion and a hand; the support device comprising:
an upper arm support surface adapted to support the upper arm portion;
a lower arm support surface adapted to support the lower arm portion;
a hand support surface adapted to support the hand;
an attachment mechanism for attaching the device to the table;
a first joint disposed between the attachment mechanism and the upper arm support surface for permitting controlled motion of the upper arm support surface relative to the attachment mechanism;
a second joint disposed between the upper arm support surface and the lower arm support surface for permitting controlled motion of the lower arm support surface relative to the upper arm support surface;
a third joint disposed between the lower arm support surface and the hand support surface for permitting controlled motion of the hand support surface relative to the lower arm support surface.

2. The support device of claim 1 wherein each of the first, second and third joints include a releasable locking mechanism for releasably locking the respective first second and third joints in place.

3. The support device of claim 1 wherein the first joint permits controlled rotary motion of the upper arm support surface relative to the attachment mechanism about a substantially vertical axis relative to the patient.

3. The support device of claim 1 wherein the second joint permits controlled rotary motion of the lower arm support surface relative to the upper arm support surface about a generally horizontal axis relative to the patient.

4. The support device of claim 1 wherein the third joint permits controlled rotary motion of the hand support surface about a generally horizontal axis relative to the patient.

5. The support device of claim 1 wherein:
the first joint permits controlled rotary motion of the upper arm support surface relative to the attachment mechanism about a substantially vertical axis relative to the patient;
the second joint permits controlled rotary motion of the lower arm support surface relative to the upper arm support surface mechanism about a generally horizontal axis relative to the patient; and
the third joint permits controlled rotary motion of the hand support surface about a generally horizontal axis relative to the patient.

6. The support device of claim 5 wherein each of the first, second and third joints include a releasable locking mechanism for releasably locking the respective first second and third joints in place.

7. The support device of claim 1, wherein one of the support surfaces includes a generally u-shaped arm rest.

8. The support device of claim 7, wherein the generally u-shaped arm rest includes gel-foam padding for engaging the patient.

9. The support device of claim 1, wherein the attachment mechanism comprises a combination suction cup and clamping mechanism for removable securing the support device to the table.

10. The support device of claim 9, including a suction pump for providing controlled suction to the suction cup.

11. The support device of claim 1 including a warming sleeve adapted to wrap about the patient's arm.

12. For a patient positioned on a table in a supine position, a radial artery procedure support device for supporting the patient's arm such as during performance of a radial artery procedure by an operator positioned opposite the patient's arm, the arm comprising an upper arm portion, a lower arm portion and a hand; the support device comprising:
an upper arm support surface adapted to support the upper arm portion;
a lower arm support surface adapted to support the lower arm portion;
a hand support surface adapted to support the hand;
an attachment mechanism for attaching the device to the table;
a first joint disposed between the attachment mechanism and the upper arm support surface for permitting controlled motion of the upper arm support surface relative to the attachment mechanism, wherein the first joint comprises a first joint portion and a second joint portion, wherein the first joint portion permits controlled rotary motion of the upper arm support surface relative to the attachment mechanism about a substantially horizontal axis relative to the patient, and the second joint portion permits controlled lateral and rotary motion of the upper arm support surface and the attachment mechanism; and
a second joint disposed between the lower arm support surface and the hand support surface for permitting controlled motion of the hand support surface relative to the lower arm support surface.

13. The support device of claim 12 wherein each of the first and second joints include a releasable locking mechanism for releasably locking the respective first and second joints in place.

14. The support device of claim 12, wherein one of the support surfaces includes a generally u-shaped arm rest.

15. The support device of claim 14, wherein the generally u-shaped arm rest includes gel-foam padding for engaging the patient.

16. The support device of claim 12, wherein the attachment mechanism comprises a combination suction cup and clamping mechanism for removable securing the support device to the table.

17. The support device of claim 16, including a suction pump for providing controlled suction to the suction cup.

18. The support device of claim 12 including a warming sleeve adapted to wrap about the patient's arm.

19. A warming sleeve for warming a patient's arm during a radial artery procedure, the warming sleeve adapted to wrap about the patients arm and having an opening to provide access there-through to the patient's radial arteries and veins.

20. The warming sleeve of claim 18 comprising three layers of fabrin, and a heating mechanism disposed with a middle layer of the three layers.
